(19) United States
(12) Patent Application Publication
(10) Pub. No.: US 2011/0130647 A1
(43) Pub. Date: Jun. 2, 2011

(54) System And Method Using Coupler-Resonators For Electron Paramagnetic Resonance Spectroscopy

(75) Inventors: Harold Swartz, Lyme, NH (US); Piotr Lesniewski, West Lebanon, NH (US); Hong Bin Li, Morrisville, NC (US)

(73) Assignee: The Trustees of Dartmouth College noneprofit corporation

(21) Appl. No.: 13/056,927

(22) PCT Filed: Jul. 30, 2009
(86) PCT No.: PCT/US2009/052261

§ 371 Date: Jan. 31, 2011

Related U.S. Application Data
(60) Provisional application No. 61/085,337, filed on Jul. 31, 2008.

(51) Int. Cl. A61B 5/055; G01R 33/44

(52) U.S. Cl. 600/421; 324/307

(57) ABSTRACT

A coupler-resonator for electron paramagnetic resonance (EPR) spectroscopy in subjects has a wire loop formed into a coupling loop, a central transmission portion, and sensor loops. The sensor loops hold EPR sensor materials and are coated with biocompatible plastic. The coupler-resonator is implanted in a subject, the subject in a nonuniform magnetic field with a pickup coil for RF response measurement apparatus near the subject's skin and inductively coupled to the coupling loop. Resonances are measured at multiple sensor loops distinguished by sweeping magnetic field or radio frequency. A biopsy sampler has an outer needle with sensor loop and a central sampling needle with cavity for biopsy samples and EPR sensor material. A device for EPR of fingernails has sensor loops in a partial glove for holding loops next to fingertips. A device for EPR of teeth has sensor loops in plastic chips that can be held between the teeth.

### RELATED APPLICATIONS

This application claims priority to U.S. Provisional Patent Application Ser. No. 61/085,337, filed Jul. 31, 2008, the disclosure of which is incorporated herein by reference.

### GOVERNMENT RIGHTS

This work was done with U.S. Government funding through National Institutes of Health grant numbers P41 EB002032 and PO1 EB2180. This research was also funded through Defense Advanced Research Projects Agency grant numbers HR0011-08-C-0022 and HR0011-08-C-0023. In consequence thereof, the United States Government has certain rights in the herein disclosed inventions.

### FIELD

The present document relates to the field of electron paramagnetic resonance (also known as electron spin resonance) spectroscopy as applied to biomedical research and medicine.

### BACKGROUND

While most molecules have paired electrons in consequence of covalent bonding, some molecules—including free radicals—have electrons that are not paired. Paired electrons have opposite spins (Mₛ=+/−½) that cancel out net magnetic moments and reduce interaction with external fields. Unpaired electrons, however, have spins that can interact with magnetic fields.

Unpaired electrons in molecules will resonate in a magnetic field. Electron Paramagnetic Resonance Spectroscopy (EPR), sometimes known as Electron Spin Resonance Spectroscopy, takes advantage of this effect to quantify and determine environments of the unpaired electrons. This is done by applying a magnetic field to a substance, which may be within a subject, to align spins of unpaired electrons in the substance. Once spins are aligned, a response of the spins of unpaired electrons in the substance to radio-frequency electromagnetic radiation at and near a resonant frequency is measured. The resonant frequency is often dependent on the local environment of the unpaired electrons in the molecule as well as the applied magnetic field. The resonance results in such effects as a spike at a particular frequency in a radio-frequency absorption spectrum of the substance in a magnetic field; the particular frequency depends on the strength of the magnetic field.

Unpaired electrons are naturally found in small quantities in chemicals, such as free radicals, that are found in biological materials.

Lithium Phtalocyanine crystals are known to have unpaired electrons. These unpaired electrons have local environments that change with local oxygen concentration. Lithium Phtalocyanine (LiPc) crystals in a constant magnetic field therefore have a broader absorption EPR resonance in high oxygen environments than in low oxygen environments.

Oxygen O₂ molecules themselves have two unpaired electrons in partially occupied orbitals (the overall energy is lower than if the electrons were in the same orbital; the latter condition is termed singlet oxygen) but because of the strong interactions of these unpaired spins with each other, the EPR resonance is very broad and usually not detected. Similarly, oxygen free radicals are at very low concentrations in tissue and are not usually measurable with this technique. The EPR resonance measured when determining oxygen concentrations in this technique is that of unpaired delocalized electrons in the LiPc crystals; this resonance is affected by magnetic interactions with the unpaired electrons of oxygen.

This interaction of LiPc with the unpaired electrons of oxygen has been utilized to measure the partial pressure of molecular oxygen in various tissues. The LiPc is quite unreactive, and therefore, there is little or no reaction of the tissue to its presence, even after months or years.

Similarly, some formulations of India ink have been reported as providing an oxygen-sensitive EPR resonance that can be used to monitor oxygen concentrations in skin.

Unfortunately, at typical EPR system operating frequencies and magnetic field strengths, most systems have difficulty sensing EPR spectra from such crystals when the crystals are located at depths of more than about one centimeter in tissue. While this is adequate for many studies in mice, it represents a serious limitation when it is desired to use EPR in larger animals or in humans.

It should be noted that EPR is not nuclear magnetic resonance (NMR). In EPR, it is unpaired electrons that resonate, while in NMR or its imaging variation Magnetic Resonance Imaging (MRI), it is nuclei with net spins that resonate. Magnetic field strengths differ by several orders of magnitude between typical NMR and EPR spectrometers. In biomedical research and in medical applications, MRI is typically used to examine resonances of the hydrogen nuclei of water; these are found at up to about 100 molar concentration in mammalian tissues. EPR typically cannot directly observe the concentrations of unpaired electrons that occur in living systems and therefore sensor molecules with unpaired electrons are usually added. One of the few exceptions to this is the radiation-induced unpaired electrons in bone, teeth, and keratin-rich materials that are detectable by EPR in vivo and are potentially useful for measuring total absorbed radiation dose. The challenges and applications of EPR are therefore considerably different from those of NMR and MRI.

### SUMMARY

An implantable resonator and coupling device for electron paramagnetic resonance spectroscopy in living animals or human subjects has a conductive wire loop of non-ferrous material. The wire loop is formed into a coupling loop, a central transmission portion, and a sensor loop. The sensor loop holds an electron paramagnetic resonance sensor material. The device is coated with a biocompatible plastic.

The implantable resonator and coupling devices are used in a system for measuring parameters in a living animal or human subject. A multiple-sensor-loop version of the device is implanted in a subject; the subject is placed in a nonuniform magnetic field with a pickup coil near its skin for measuring a radio frequency response. The pickup coil is inductively coupled to the coupling loop. Resonances are measured at the each of the multiple sensor loops, the loops may be distinguished by changing either the magnetic field or the radio frequency.

A biopsy sampling device has a nonconductive outer needle having a conductive sensor loop. The device also has a nonconductive central sampling needle with a cavity for holding a sample of a biological material and an electron paramagnetic resonance sensor material.

A device for EPR of fingernails has sensor loops in a partial glove for holding the loops adjacent to fingertips. A device for EPR of teeth has sensor loops in a plastic chip that can be held between the teeth. The devices for EPR of teeth and fingernails are used for determine EPR resonances that provide a measure of cumulate radiation exposure of a subject, a measure which may be of use in triage following a release of radioactive materials or a nuclear attack.

### BRIEF DESCRIPTION OF THE DRAWINGS

FIG. 1 is a schematic of a PRIOR ART implantable coupler-resonator for EPR sensing in biological or medical applications.

FIG. 2 illustrates a simplified implantable coupler-resonator for EPR sensing in biological or medical applications.

FIG. 3 illustrates an implantable coupler-resonator for EPR sensing having multiple sensor loops on multiple leads.

FIG. 4 illustrates an implantable coupler-resonator for EPR sensing having multiple sensor loops on a single lead.

FIG. 5 illustrates an implantable coupler-resonator for EPR sensing having multiple sensor loops in forklike arrangement.

FIG. 6 is a thresholded and edge-enhanced photograph of an implantable coupler-resonator for EPR sensing having multiple sensor loops in forklike arrangement and a pigtail for tuning the coupler-resonator and as used in the experiment of FIGS. 14-15.

FIG. 7 is a cross sectional diagram illustrating the implantable coupler-resonator implanted to allow repeated monitoring of oxygen levels during divided-dose radiotherapy of a tumor in liver of a subject.

FIG. 8 is a cross sectional diagram illustrating the subject in EPR magnet for monitoring of oxygen levels in tumor.

FIG. 9 is a cross sectional diagram of an EPR-guided biopsy sampling device.

FIG. 10 is a side view of the trocar of the EPR-guided biopsy sampling device showing the coupling loop.

FIG. 11 is a side view of a sensing loop device clenched between teeth of a subject.

FIG. 12 is a top view of a sensing loop device suitable for sensing EPR resonances in enamel of the teeth and fingernails.

FIG. 13 is a view of a coupler-resonator for EPR resonances in fingernails.

FIG. 14 illustrates placement of the coupler-resonator in a rat brain for studies of ischemia and reperfusion injury following experimental middle cerebral artery occlusion.

FIG. 15 illustrates signals obtained using the coupler-resonator implanted in rat brain as illustrated in FIG. 14.

FIG. 16 illustrates placement of the coupler-resonator in a rabbit heart for studies of ischemia and reperfusion injury following experimental coronary artery occlusion.

### DETAILED DESCRIPTION OF THE EMBODIMENTS

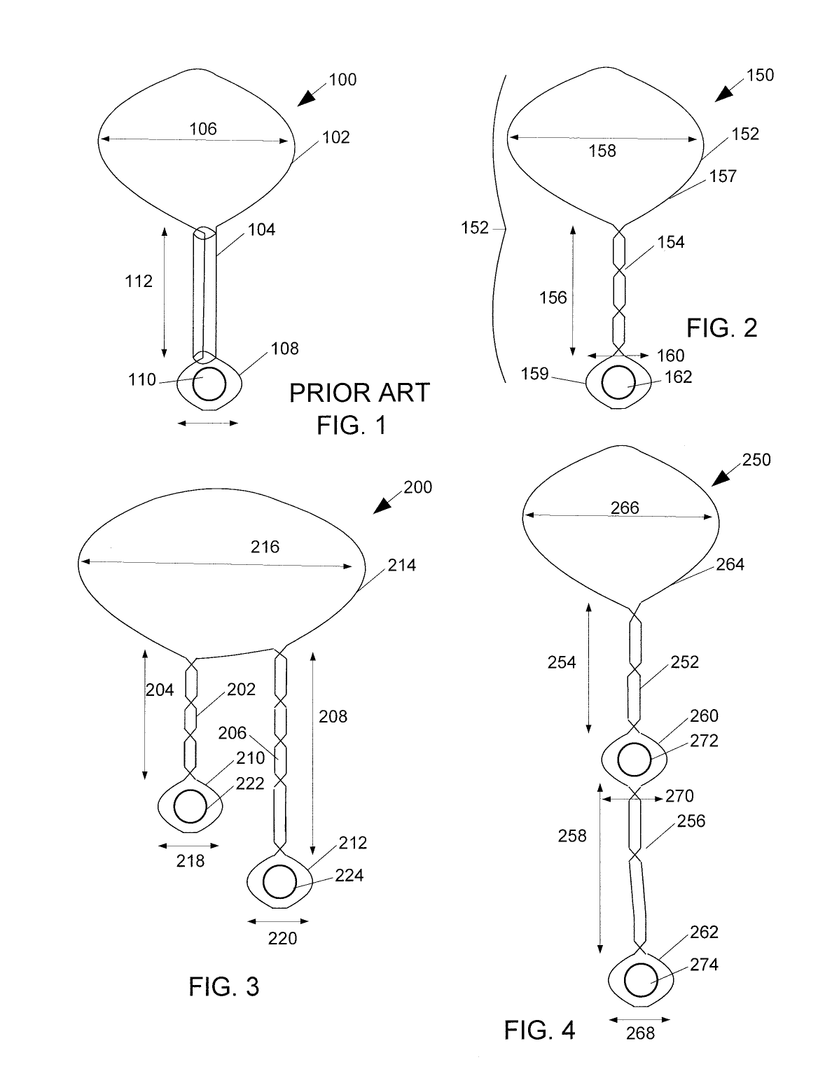

The PRIOR ART device 100 of FIG. 1 was previously tried and presented in 2004. In this device, a coupling loop 102 was made of enameled copper wire. One end of the loop 102 was carefully soldered to the center conductor of a miniature coaxial transmission line cable 104, the other end of the loop was soldered to the shield of the coaxial cable 104. The loop 102 had a diameter 106 of five millimeters. At the opposite end of the coaxial cable, the center conductor of the coaxial cable 104 was formed into a sensing loop 108 of approximately six-tenths of a millimeter in diameter and soldered to the shield, the sensing loop 108 formed about a sensor capsule 110. The coaxial cable was sized according to the equation length (112)=[π−arctan(ωL/Z0)]/β, where L is the inductance of the larger loop, Z0 is the characteristic impedance of the transmission line, β is the phase constant 2π/λ, and ω is the operation angular frequency 2π f. The soldered connections are then coated with a plastic material to avoid corrosion, but no biocompatible plastic coating was used. The device of FIG. 1 was tested implanted in an animal, but was not used on teeth or fingernails, and proved too stiff for use in measurements in highly mobile organs such as heart.

Since 2004, further experimentation has shown that use of coaxial cable, as in the device of FIG. 1, is not essential. In the coupler-resonator 150 of FIG. 2, a wire loop 152 formed from enameled copper or silver wire of size gauge 34 to 36, having a wire diameter with enamel of approximately 0.13 to 0.16 millimeter. It is suggested that wire of ferrous materials, such as stainless steel, be avoided to prevent interference with the magnetic fields of the EPR apparatus. In an alternative embodiment, the wire loop is formed from stranded wire. The loop is formed by soldering ends of the wire to form a mechanical and electrical connection. In an alternative embodiment, 350 of FIG. 6, the ends of the wire are twisted about each other to form a mechanically-connecting pigtail that closes the loop and is an effective capacitive element, and solder is not applied.

The wire loop is pinched together along a central portion of arbitrary length 156 to form a transmission line portion; in some embodiments this central transmission line portion is formed with wires of opposite sides of the loop parallel to and adjacent to each other and retained by a gas-permeable plastic coating. In most embodiments, this central transmission line portion is twisted 154 both to mechanically secure the opposite strands together and to limit electromagnetic coupling to a coupling loop 157. The central transmission line portion has arbitrary length 156 ranging from less than one centimeter to more than fifteen centimeters in length—length is chosen as appropriate for an application of the coupler-resonator. Remaining portions of the wire loop 152 form an untwisted coupling loop 157 typically of approximately ten millimeters diameter 158, loops of between five and fifteen millimeters in diameter are expected to work. In some applications a coupling loop of up to twenty millimeters diameter 158 may be used. Remaining portions of the wire loop also form sensor loops 159 of diameter 160 approximately half to one millimeter. A capsule 162 of EPR sensing material such as LiPc is retained in the sensor loop 159 within an envelope formed by a gas permeable coating.

The loop 152 is coated with a biocompatible plastic selected from fluorocarbon and dimethylsiloxane materials. The coupler-resonator of FIG. 2 has been found effective and easier to make than the prior device of FIG. 1.

In an embodiment, the capsule 162 of EPR sensing material is formed by dipping the sensing loop 159 in a suspension or solution of the biocompatible, gas-permeable, plastic to formn a film across the loop 159, placing sensor material such as LiPc on the film, and applying additional biocompatible plastic to coat the sensor material and secure it to the loop 159. In an embodiment, the coupling loop 157 is then bent at a 90-degree angle to the twisted central portion.

In alternative embodiments, other and additional bends may be applied, for example, in one embodiment the twisted central portion is directed from the periphery of the coupling loop to the center of the coupling loop, at which point a 90-degree bend directs the central portion along an axis of the coupling loop. The length 156 of the twisted portion is predetermined for each device; however, this length is not determined from the wavelength of electromagnetic radiation used in EPR and may be virtually any length in the range from one to fifteen centimeters.

Alternative EPR sensor materials may be used within the sensor loops. In particular, LiPc, as well as some India inks, charcoals, wood char, as well as some nitroxides and trityls have demonstrated sensitivity to oxygen in tissue and may serve as an EPR sensor material for sensing oxygen levels. Similarly, some dithiocarbamates have demonstrated sensitivity to nitric oxide in tissue and may serve as an EPR sensor material for sensing nitric oxide levels in tissue. Some nitrone spin-trap materials, including phenyl-N-tert-butylnitrone (PBN) and 5,5-dimethyl-1-pyrroline N-oxide (DMPO), 5-diethoxyphosphory-5-methyl-1-pyrroline N-oxide (DEMPO), α-(4-pyridiyl-1-oxide)-N-tert-butyl nitrone (4-POBN) have demonstrated sensitivity to other reactive oxygen and reactive nitrogen species including some free radicals and may serve as an EPR sensor material. Yet other materials, such as nitroxide compounds having amino or acid moieties near the nitroxide group, are known to have EPR resonances that are sensitive to pH and may serve as an EPR sensor material responsive to pH. Some other nitroxides have EPR spectra that smear under mechanical motion; others have EPR spectra that are sensitive to concentrations of sulfhydryl (SH) groups. Similarly some other nitroxides have hyperfine spectra that change with membrane potential of nearby cell membranes; since muscle and neurological tissues, among other tissue types, have membrane potentials that change radically as they function, these nitroxides may serve as an EPR sensor material useful in studying such tissues. It is expected that, with biocompatible plastic coatings permeable to the appropriate target molecules, the implantable coupler-resonator herein described will function with most of the EPR sensor materials discussed in “Measurements in vivo of parameters pertinent to ROS/RNS using EPR spectroscopy,” Nadeem Khan and Harold Swartz, Molecular and Cellular Biochemistry 2341235: 341-357, 2002. With appropriate EPR sensor materials, the present coupler-resonators are expected to be able to monitor selected pH, Oxygen concentrations, Nitric oxide concentrations, SH (sulfhydryl) concentrations, and other entities of interest in biomedical research and medicine.

Embodiments of the present coupler-resonator have been tested in the L-band near 1.2 GHz, and, with appropriate magnetic fields. Other embodiments are believed operable at other frequencies including S-band near 2.4 GHz and potentially at X-band frequencies.

Since nitric oxide has been demonstrated to be a neurotransmitter as well as a vasodilator, and is difficult to study with prior techniques, use of dithiocarbamates and other nitric oxide-sensitive EPR sensor materials with the present invention for monitoring of in-vivo nitric oxide levels is of particular interest in biomedical research.

Many organs of human subjects and experimental animals move relative to other organs, or change shape, during everyday activities; these organs are known as mobile organs. Mobile organs include the heart, muscles, and the hollow organs of the digestive system. The embodiments of FIGS. 2-5 have much more flexible transmission line portions than the prior art coaxial cable of FIG. 1. The loop may also be made of a stranded wire for still greater flexibility. It is expected that the present coupler-resonators will be far better for use with sensor loops and EPR capsules implanted in mobile organs than prior devices.

The embodiment 200 of FIG. 3 is formed similarly to that of FIG. 2, except that after forming the wire loop, it is divided and twisted into a first 202 twisted portion of a first length 204, a second 206 twisted portion of a second length 208 which may or may not be the same as the first length, a first sensor loop 210, a second sensor loop 212, and a coupling loop 214. The coupling loop 214 diameter 216 is about one centimeter as with the embodiment of FIG. 2, and the sensor loops 210, 212 are of diameter 218, 220 approximately half to one millimeter. A capsule 222, 224 of an EPR sensing material, such as LiPc and chosen as appropriate for studies to be performed with the coupler-resonator, is retained in each sensor loop 210, 212 as previously discussed.

The embodiment 250 of FIG. 4 is formed similarly to that of FIG. 2, except that after forming the wire loop, it is divided and twisted into a first 252 twisted portion of a first length 254, a second 256 twisted portion of a second length 258, which may or may not be the same as the first length, a first sensor loop 260, a second sensor loop 262, and a coupling loop 264, and first and second twisted portions 252, 256. The coupling loop 264 diameter 266 is about one centimeter as with the embodiment of FIG. 2, and the sensor loops 260, 262 are of diameter 268, 270 approximately between half and one millimeter. A capsule 272, 274 of EPR sensing material, such as LiPc, is retained in each sensor loop 260, 264 as previously discussed.

While the embodiment of FIG. 4 is illustrated with two sensor loops, embodiments have been constructed with other numbers of sensor loops.

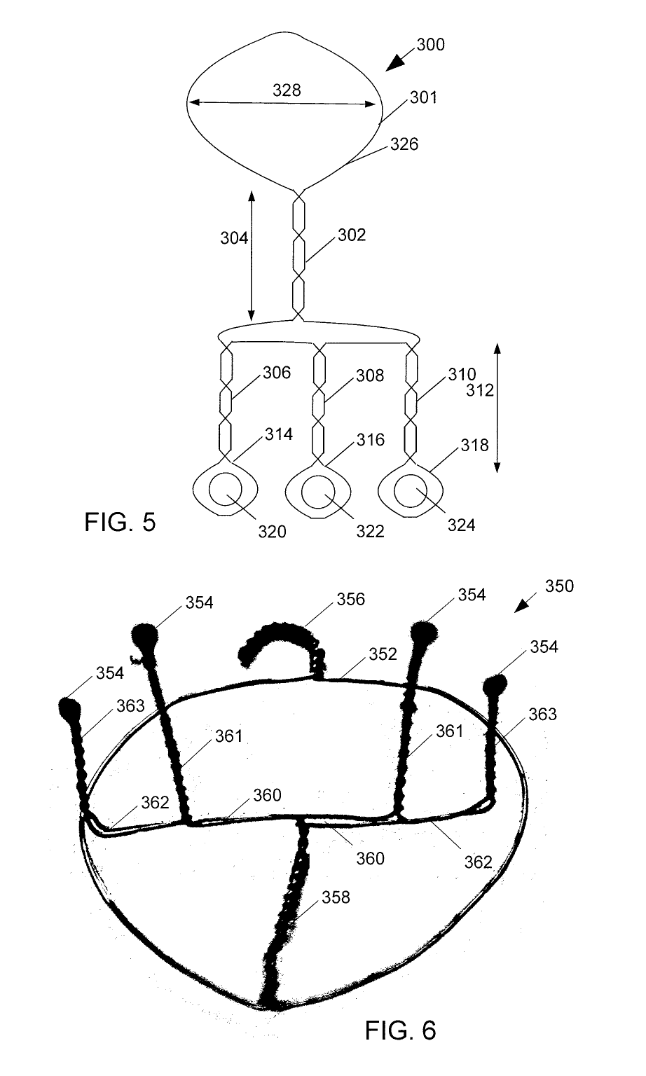

The embodiment 300 of FIG. 5 is formed similarly to that of FIG. 2, except that after forming the wire loop 301, it is divided and twisted into a first 302 twisted portion of a first length 304, then further divided into a second, third, and fourth 306, 308, 310 twisted portion, or tine portion, of a second length 312 which may or may not be the same as the first length. The tine portions 308, 310, may be of unequal length 312, and lengths 304, 312, need not be calculated based upon the wavelength of the resonance. The wire loop 301 is further formed into first, second, and third sensor loops 314, 316, and 318. A capsule 320, 322, 324 of EPR sensing material such as LiPc is retained in each sensor loop 314, 316, 318 as previously discussed. The coupling loop 326 diameter 328 is about one centimeter as with the embodiment of FIG. 2, and the sensor loops 314, 316, 318 are of diameter approximately half to one millimeter.

While the embodiment of FIG. 5 is illustrated with three sensor loops and EPR sensing capsules, embodiments have been constructed and tested with other numbers of sensor loops including two, four, and five sensor loops. It is believed that other numbers of sensor loops will function. Alternative embodiments resembling that of FIG. 5 may also have tine portions 306, 308, 310 of unequal lengths.

The embodiments of FIGS. 2-5 may be formed further as appropriate for the application, for example there may be a 90-degree bend at the junction of coupling loop 157, 264, 326 and the twisted transmission line portion 154, 252, 302. Such a bend permits the coupling loop to lie flat beneath skin of an animal or subject while allowing the transmission line portion to extend into deeper tissues to a point of interest. In use, the coupling loop is inductively coupled to a pickup coil of resonance-measuring apparatus, the pickup coil may be placed on or near skin over the coupling loop.

The embodiment 350 of FIG. 6 is also formed from a wire loop formed by twisting ends of a length of insulated wire together to form a capacitive element referred to as a pigtail 356. The wire loop is divided into a coupling loop 352, a twisted transmission line portion 358, four sensor loops 354, and a pigtail 356 portion. The transmission line portion 358 has a portion coplanar with, and extending from the outer rim of the coupling loop 352 towards a center of, the coupling loop 352. At approximately a center of the coupling loop 352, the transmission line portion 358 divides into a first and a second arm 360, also twisted, which are have proximal portions also coplanar with the coupling loop 352. First and second aim 360 each divide into a first and second distal portion 361, 362, with the first distal portion 361 extending vertically at 90 degrees from the associated arm 360, and the second distal portion 362 extending collinear with arm 360. First and second atm 360 have approximately a 90-degree bend such that each arm 362 has a distal portion 363 approximately parallel to an axis of the coupling loop and to first distal portions 361, and extending to the sensor loops 354. The sensor loops 354 have EPR sensor material in capsules within the loop, although these capsules can not be seen separately in the figure. It has been found that the length and number of turns of pigtail portion 356 may be adjusted to better tune resonance of the embodiment 350 for improved performance. The performance of the device, including the resonant frequency of the device and the associated quality factor, can be monitored using a network analyzer with appropriate inductively coupled probe as the length and/or twist of the pigtail is adjusted. A device similar to that illustrated in FIG. 6 was inverted and implanted into a rat such that the coupling loop 352 lay under the rat's scalp, and the sensor loops 354 were in brain.

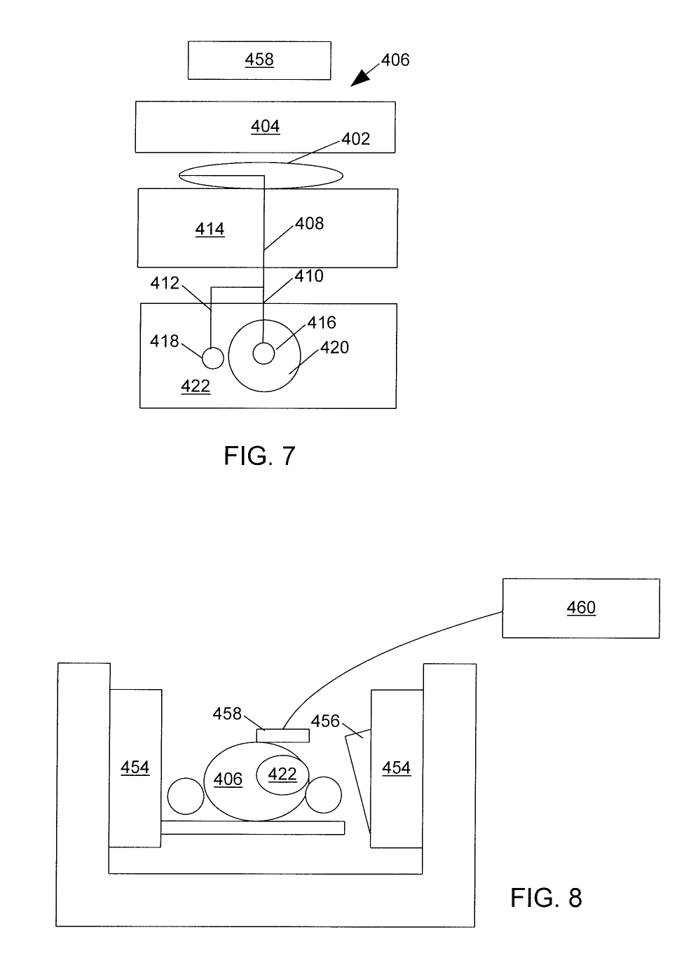

When the implantable sensors of FIGS. 2, 3, 4, 5, and 6 are used, they are implanted with the coupling loop 402 (FIG. 7) beneath the skin 404 of an animal or human subject 406. Twisted transmission lines portions, such as twisted portions 408, 410, 412 are routed through tissues not of interest for the study intended, such as abdominal muscle 414, but the EPR sensing capsules and sensor loops 416, 418 are placed in tissues of interest, such as by example and not limitation in a liver tumor 420 and as a control in liver stroma 422. Many other tissue types may be of interest, including brain tissue. This implantation may, but need not, be done at the time of tumor debulking surgery.

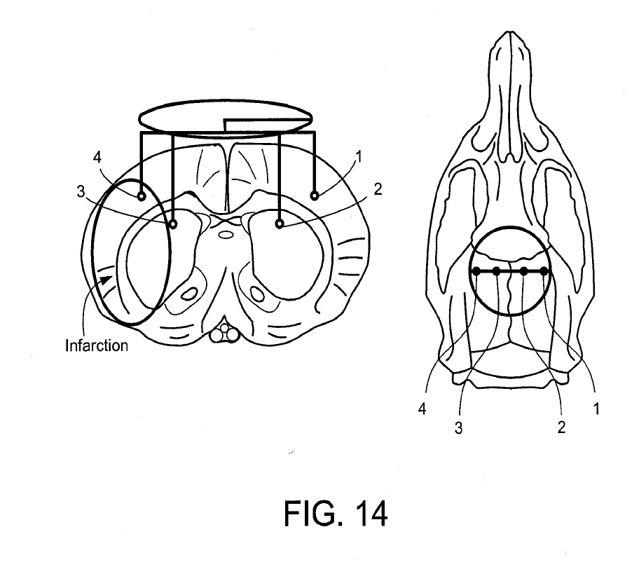

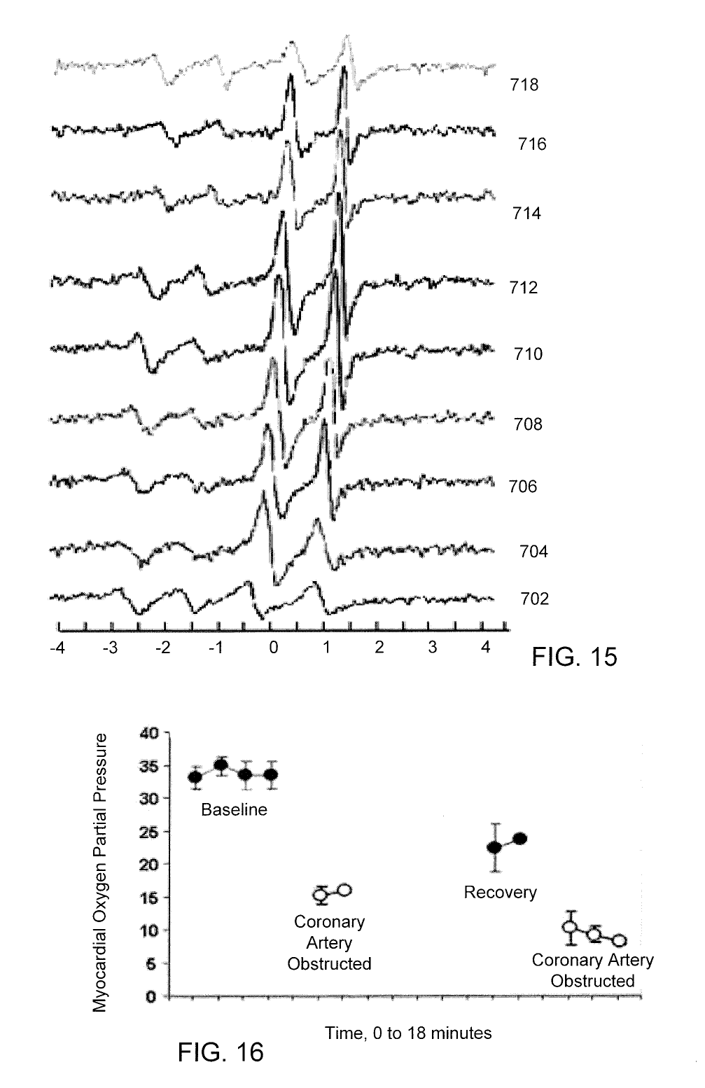

In an experiment, a device similar to that illustrated in FIG. 6 has been inverted and implanted in the brain of a rat, see also the discussion with reference to FIGS. 14-15 below. A first sensor loop 354 was placed in a tumor in one hemisphere of the rat brain, the tumor being derived from an F98 rat glioma cell line. A second sensor loop 354 of the device was placed in normal tissue at a position in the other hemisphere of the rat's brain at a position anatomically approximately equivalent to the position of the first sensor loop. The coupling loop 352 was placed beneath the scalp of the rat. Measurements of static oxygen levels and of a dynamic response to breathing oxygen-enriched air were taken every two days for twenty days following the implantation. In this experiment, significant differences in static and dynamic response oxygen levels were observed between the tumor tissue and the normal tissue. In one such rat at day 17, static oxygen partial pressure in the glioma was measured as approximately twenty millimeters of mercury, rising to a level of less than forty when breathing oxygen enriched air; while static oxygen partial pressure in normal tissue was approximately sixty millimeters of mercury, rising past one hundred fifty millimeters of mercury after fifteen minutes of breathing oxygen-enriched air. Significantly lower oxygen levels were observed in tumor of another rat, also showing significantly reduced response to breathing oxygen-enriched air as compared to normal tissue. It is believed that these differences are due to differences in perfusion between tumor and normal tissue, and it is expected that the device can provide static and dynamic oxygen-level monitoring within tissues and tumors of other species, including primates and human subjects.

When it is desired to monitor parameters, such as oxygen or nitric oxide concentrations in the tissues of interest, without further invasive surgery, the animal or subject 406 (FIG. 8) is placed in a nonuniform magnetic field (not shown) between poles 454 of a magnet. The nonuniform magnetic field may be formed with trimming plates 456 or with trimming coils (not shown) as known in the art of magnets. With reference also to FIG. 7, a pickup coil 458 is placed over the skin 404 (not labeled in FIG. 8) of the animal or subject 406. An apparatus 460 for measuring a radio frequency response is connected to the pickup coil 458, this apparatus 460 provides pulses of radio frequency energy at and near frequencies where an EPR response is expected from the EPR sensing capsules, and observes for resonance.

In one embodiment, this apparatus 460 maintains a constant frequency and a sweep of magnetic field strength is performed. In a second embodiment, the magnetic field strengths are held constant, and the frequency of apparatus 460 is swept through a range. In both embodiments, each EPR sensing capsule resonates only when the magnetic field strength, material properties, and frequency satisfy the criteria for resonance—since this occurs at different times for each sensor loop and capsule in each sweep because of the nonuniform magnetic field, the responses of each of the EPR sensing capsules are readily distinguished. A computer of the system then calculates measured parameters, such as oxygen concentration, for each sensor loop and EPR sensing capsule individually, the calculation is performed according to a calibration table for the EPR sensing material.

A typical and clinically important use of the device is to follow the partial pressure of oxygen in a tumor and adjacent normal tissue during a course of fractional radiation therapy, chemotherapy or combined radiation and chemo therapies. The response to radiation therapy is very dependent on the oxygen concentration in the tumor up to about 25 torr (above that level the response is constant). During the course of radiation and/or chemotherapy the partial pressure of oxygen in the tumor varies with time after each dose, it may increase, decrease, or both from baseline levels. The information obtained from the implantable coupler-resonators therefore can be used by the clinician to time delivery of doses of radiation and/or chemotherapy so that these doses are given under the most favorable partial pressures of oxygen that occur in that particular patient's tumor. Administering radiation and/or chemotherapy doses at these favorable times increases effectiveness of these treatments against tumor tissue, thereby increasing the therapeutic ratio.

As heretofore described, each implantable coupler-resonator device may have multiple sensor loops, and the EPR response of each of these sensor loops may be determined individually through use of a non-uniform magnetic field. Since the magnetic field at each sensor loop is slightly different, and the EPR resonance frequency is dependent on the magnetic field strength, the resonances at each sensor loop are at slightly different frequencies. Tuning of a radio frequency resonance-measuring device or sweeping of the magnetic field to select particular sensor loops may be accomplished within milliseconds; this is brief enough that resonances at each of multiple sensor loops may be read sequentially at what is effectively the same time in a biological system. Since tuning the radio frequency device or sweeping the magnetic field can be repeated rapidly, and the EPR sensor material at each sensor loop can respond rapidly to concentration changes because of the small size of each loop and thin coating of each sensor material capsule, measurements at each sensor loop may be repeated rapidly enough to provide realtime dynamic monitoring of oxygen, nitric oxide, or other target substance levels in biological tissues. Each sensor loop and associated encapsulated EPR sensor material is responsive to its target substance at its discrete location within the subject.

This capability for monitoring multiple sensors may be advantageously used during monitoring of chemotherapy. For example, a five-sensor-loop coupler-resonator may be implanted in a subject, with sensor loops containing oxygen-sensitive material placed in three separate metastatic tumor nodules in liver to provide oxygen concentration in tumor measurements, and two placed in nearby liver stroma to provide oxygen concentrations in normal liver stroma. Oxygen levels are measured separately at each of the five sensor loops; these measurements are used to time subsequent doses of radiation and/or chemotherapy for maximum effectiveness in all three tumor nodules while timing the doses to minimize damage to surrounding normal liver stroma.

Further, multiple coupler-resonators may be implanted in the same subject, permitting use of large numbers of sensor loop locations.

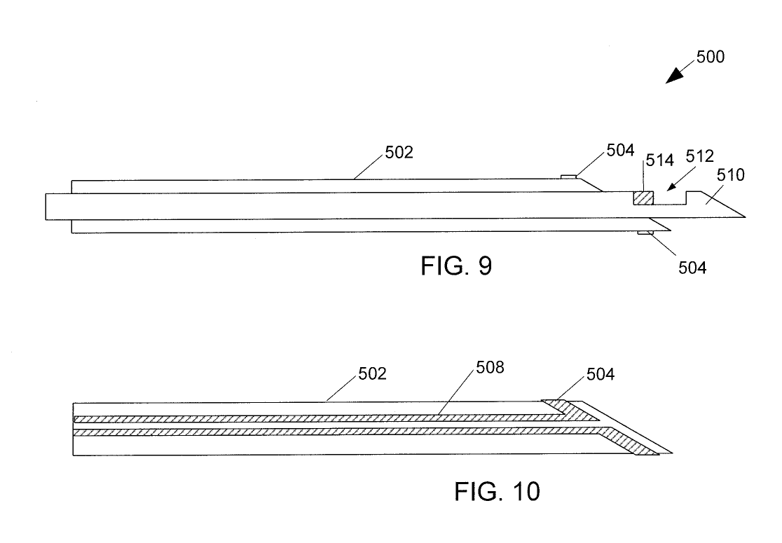

It is known that it is not always easy to ensure that biopsy samples are actually taken from a tissue of interest, such as a tumor. Oxygen concentrations of tumor and normal organ stroma may differ; indeed many tumors have necrotic cores due to hypoxic conditions within the tumors. In order to help ensure that a biopsy sample is taken from tumor tissue and not from normal stroma, an EPR biopsy sampling device may be used. This device is illustrated in FIGS. 9 and 10. This device 500 has a nonconductive ceramic or glass hollow outer needle 502 of about 16 gauge diameter. Deposited on the outer surface of one side of the needle is a pair of closely spaced conductors 508, preferably of a thick-film conductive silver-frit type or similar material that is fired into the needle. In an embodiment, thickness of the frit is approximately 2 microns, spacing between the conductors is 10 microns, and conductor width is about 10 microns. This pair of conductors 508 forms a non-twisted microstrip transmission line. Near the sharpened beveled end of outer needle 502, and continuous with conductors 508, the thick-film conductive material forms a single or double turn sensor loop 504. In an alternative embodiment, the conductive material is a thin-film nonmagnetic conductor deposited on the needle and covered with a nonconductive protective layer.

The device 500 also has an inner stylette 510 having a sharpened end. Near the sharpened end of stylette 510 is a cavity 512 for capturing a biopsy sample, and at one end of the cavity is a small quantity of EPR sensor material 514 such as LiPc encapsulated in a gas permeable material.

In use, the subject is placed between poles of a magnet, and conductors 508 are coupled to apparatus for measuring an EPR response of the EPR sensor material. The stylette is inserted into the outer needle such that the EPR sensor material 514 is close to the sensor loop 504 and exposed to tissue. Oxygen concentration is monitored by monitoring the EPR response as the sampling device is inserted into the subject. When a change of oxygen concentration is observed that indicates that the stylette's opening 512 is likely within a tumor or other inclusion in an organ, stylette 510 is withdrawn to entrap a sample of the tumor within cavity 512. The sample is removed, placed in a sample vial, and the stylette re-inserted into the outer needle. The device 500 may then be repositioned to take additional samples from other positions in the organ.

As LiPc and other EPR sensor materials are often also MRI contrast agents that can be readily viewed in magnetic resonance imaging, in an alternative embodiment of use of the biopsy sampling device additional images are obtained with MRI techniques during insertion of the sampling device to confirm that one or more samples are taken from the tumor. Similarly, MRI imaging may be used to confirm placement of sensor loops of the coupler-resonator of FIGS. 2-5 in a tumor.

In large scale disasters, recalled history alone has proven to not always be a good indicator of exposure to toxic or radioactive materials and corresponding need for treatment. Similarly, apparent physical injuries and symptoms are not good indicators of intensity of radiation doses received by a subject. When a radiation disaster, whether by accident like Chernobyl, or weapon like Hiroshima, happens, medical care systems will likely be overloaded. To best use available resources, it is desirable to quickly sort (or triage) potential victims into categories of:
a. those who are unexposed or having received small doses such that they will recover without treatment;
b. those who have received significant doses requiring conventional, conservative, treatment, including blood transfusions and prophylactic antibiotics;
c. those who can possibly be saved by aggressive treatment such as bone marrow transplant; and
d. those who will die despite any reasonably available treatment.

It is known that ionizing radiation can lead to production of free radicals and other species having unpaired electrons. Further, such species can have long lifetimes in solid materials such as hydroxyapatite and keratin. These unpaired electrons can be measured with EPR. This effect has been used for approximate dosimitery in persons suspected of exposure to doses of ionizing radiation.

Prior techniques of measuring EPR resonances in human teeth have required either tooth removal, or use of a semirigid waveguide for coupling the apparatus for measuring radio frequency resonances to the teeth. Neither is practical for screening large numbers of potential victims during or after a mass disaster; where a resolution of one gray or better is desired. Such a resolution may require measurements from more than one tooth or more than one fingernail.

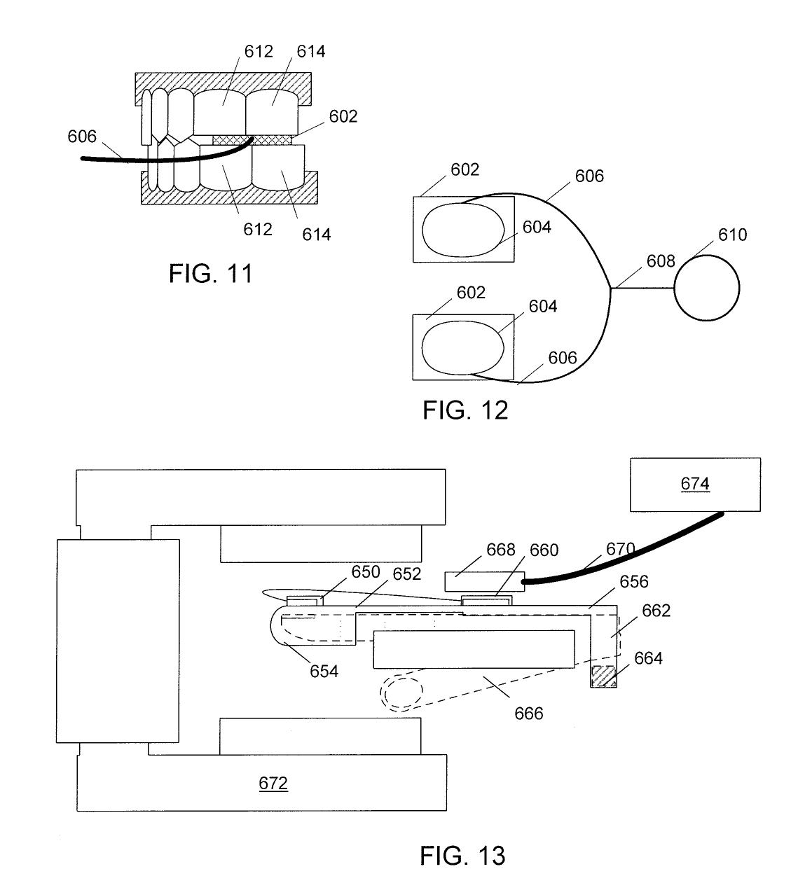

The embodiment of FIGS. 11 and 12 utilizes a thin plastic chip 602 of thickness between one and two millimeters, of width six millimeters, and of length about one centimeter. Each such plastic chip 602 has embedded within it a sensing coil 604 or sensing loop of one to two turns of copper wire and average diameter of seven millimeters. Each of the two sensing coils 604 is coupled through a twisted transmission section 606 to a common transmission section 608 and to a coupling coil or coupling loop 610 of about one centimeter diameter and having one or two turns of wire.

In use, the plastic chips are clenched between a subject's upper and lower first molars 612 and second molars 614, thereby providing coupling to enamel of these teeth, preferably four teeth on each side and eight total, for EPR sensing. The coupling loop is inductively coupled outside the subject to apparatus for measuring a radiofrequency resonance. The assembly of chips, transmission sections, and coupling loop is essentially as for the device of FIG. 2, although larger diameter wire may be used; a wire loop is formed, transmission sections are pinched together and twisted, and remaining sections form sensing and coupling loops. The sensing loops, and optionally the coupling loop, are then cast into the plastic chips, and the entire assembly coated with biocompatible plastic. The simple construction of the plastic chips and associated transmission sections and coupling loops allows for low cost manufacture and easy replacement as these components are likely to suffer eventual damage when chewed by large numbers of people.

In use, the device of FIGS. 11 and 12 is placed into the subject's mouth and clenched between the teeth. The subject's head is then inserted between poles of the magnet and resonances measured. A calibration table, based on spectral features of the EPR resonances including, but not limited to, its amplitude and shape, is used to translate measured EPR resonances into an estimate of the subject's radiation exposure. In those subjects where radiation exposure is detected, a nonuniform magnetic field may be used to separately determine resonances from left and right teeth to detect asymmetrical exposure. This use is illustrated with the use of molar teeth, but it also can be used with any teeth, so that in subjects with missing molars or extensively restored molars, EPR measurements may still be made using premolars, canines, and incisors, although different calibration tables may need to be used because of the reduced mass of enamel near the coils 604. For example, in subjects lacking molars, the plastic chips of the device of FIGS. 11 and 12 may be held between lip and upper incisors to obtain dosimetry information from the enamel of the incisors. In subjects missing only one or two of the eight first and second molars, an alternative calibration table is used to estimate exposure.

In an alternative embodiment, a coupler-resonator having a single similar chip having a single sensor coil is used. This single chip is gripped between teeth on one side of the mouth at a time. A single-sided measurement may be used for screening. Measurements with the chip gripped between right teeth, and with the chip gripped between left teeth, are summed to provide more accurate total exposure measurements than those obtainable with it gripped between teeth of one side alone. Measurements with the chip gripped between the right teeth and with the chip gripped between the left teeth are also compared to detect asymmetric exposure.

A device similar to that of FIG. 12 is also useful for detecting radiation-induced EPR resonances in fingernails and toenails. This alternative embodiment has from two to five plastic chips 602 that are, however, cast from a flexible plastic so that the coils 604 will conform to the curvature of the top of a subject's fingernails. As illustrated in FIG. 13, the plastic chips having sensing coils 604 are inserted into pockets 650 in the dorsal surface of finger cups 654 of a thin elastomeric partial glove 652 having two to five finger cups 654 attached by elastomeric straps to a backhand portion 656 positionable above the back of the subject's hand. The glove 652 holds the plastic chips near, and above, the subject's fingernails when the subject's fingertips are inserted into the finger cups 654 such that the coils 604 are near the subject's fingernails. In an embodiment, the elastomeric partial glove is made as a single piece from silicone rubber. The coils 604 are connected by transmission sections 606, 608, to a coupling loop 610 that is inserted into a pocket 660 of the backhand portion 656. A wrist strap 662 having a hook and loop fastener 664 serves to secure the partial glove 652 to a subject's hand 666. The hand, in the partial glove 652, is then inserted between poles of the magnet 672 and held close to a coupling coil 668 that is connected by a coaxial transmission line 670 to apparatus for measuring a radio-frequency resonance. The resonance is measured and an approximate radiation dose is calculated therefrom.

The device of FIG. 13 has advantage in that it is self-adjusting for many different lengths and diameters of a subject's fingers, and widths of the subject's fingers, hands and wrists.

The tooth EPR measuring device of FIG. 11 provides a measure of total radiation exposure of the subject since the teeth formed, often many years before the measurement is made. The fingernail EPR measuring device of FIG. 13 provides a measure of radiation exposure of the subject over the past few months because of growth of fingernails. It is expected that either device will provide dosimitery to a resolution of less than one gray, and will therefore provide results sufficiently accurate for triage in nuclear disaster situations.

Coronary artery occlusion, also known as heart attack, is a major killer of Americans. In such an event, an area of heart muscle that has been deprived of blood flow may be substantially damaged, or may die and be replaced by scar tissue, resulting in permanent impairment of heart function. Many such events are now treated by using medications to dissolve clots, or using mechanical devices—such as angioplasty catheters—to reopen the obstructed arteries. While both treatment methodologies often restore blood flow to the area of muscle that was deprived of blood flow before the muscle tissue dies, it has been found that some permanent damage often remains. It is known that some of this permanent damage results from oxidative damage after blood flow is restored—this is known a reperfusion injury. Some of the reperfusion injury may result from an overshoot of oxygen levels in the tissue after blood flow is restored—oxygen levels in the tissue may soar to levels considerably greater than normal for a time. It is desirable to find ways to reduce reperfusion injury following treatment of coronary artery occlusion.

Similar effects to the reperfusion injury seen after treatment of coronary artery occlusion have been observed in brain following occlusive strokes. It is desirable to find ways to reduce reperfusion injury following treatment of occlusive strokes.

A method to find a treatment to reduce reperfusion injury following treatment of occlusive events like coronary occlusion or of occlusive stroke may be to monitor oxygen levels in experimental models of occlusive events, and then try a number of medications to find a medication that inhibits the overshoot in tissue oxygenation after restoration of blood flow.

FIG. 14 illustrates placement of the coupler-resonator in a rat brain for studies of ischemia and reperfusion injury following experimental middle cerebral artery occlusion. A coupler-resonator having four or more sensor loops each having one-milligram LiPc sensor capsules was used, two of these sensor loops being placed near a portion of each cerebral hemisphere that is served by the middle cerebral artery, see 1, 2, 3, and 4 in the FIG. 14. The surgical wound was then allowed to heal.

FIG. 15 illustrates signals obtained using the coupler-resonator implanted in rat brain as illustrated in FIG. 14 when an experimental obstruction was temporarily created in the middle cerebral artery of one cerebral hemisphere. The animal was placed in a magnetic field gradient in a magnetic field to separate spectra from each of the four sensor loops, hence each trace of FIG. 14 shows four separate squiggles each corresponding to a signal from a different sensor loop. Trace 702 represents an initial trace before the obstruction was created.

Use of the coupler-resonator to monitor changes in local oxygen concentrations during the time the artery was obstructed is illustrated in traces 704, 706, 708, 710 and 712, and after removal of the obstruction and during recovery in traces 714, 716, and 718. The implantable coupler-resonator is therefore useful in monitoring local concentrations of oxygen, in brain tissue in and/or near experimental infarctions. With different sensor material, the device is also expected to be useful for monitoring nitric oxide concentrations in brain tissue in and/or near experimental infarctions; understanding nitric oxide changes in such tissues may be of importance in devising future stroke treatments because nitric oxide can serve as a potent local vasodilator and lead to excess oxygen in areas near, but not in, infracted tissue, or during post-infarction reperfusion in infracted tissue.

FIG. 16 illustrates placement of the coupler-resonator device in a rabbit heart for studies of ischemia and reperfusion injury following experimental coronary artery occlusion. In this experiment, an implantable resonator as herein described was made with thin, flexible, wire in the transmission line portion. The rabbit was anesthetized and the coupler-resonator was implanted with its coupling loop below the skin of the rabbit, with the sensor loop and LiPc capsule placed in heart muscle. The rabbit was then allowed to awaken and to heal.

At a later time, the rabbit was anesthetized again, and a temporary obstruction created in a coronary artery that is responsible for perfusing the area of heart muscle in which the sensor loop was located. A radio-frequency measuring device was coupled through skin to the coupling loop, and the rabbit was placed in a magnetic field. Local heart-muscle tissue oxygen levels were then measured at intervals both during periods in which the artery was obstructed, and during periods in which the obstruction was removed. Local oxygen levels were seen to drop during the time of the obstruction, and to rise when the obstruction was removed.

While the forgoing has been particularly shown and described with reference to particular embodiments thereof, it will be understood by those skilled in the art that various other changes in the form and details may be made without departing from the spirit and scope hereof. It is to be understood that various changes may be made in adapting the description to different embodiments without departing from the broader concepts disclosed herein and comprehended by the claims that follow.

What is claimed is:

1. An implantable resonator and coupling device for electron paramagnetic resonance spectroscopy in living mammals or subjects comprising:
a conductive wire loop of non-ferrous metal formed into a coupling loop, a central parallel-conductor transmission-line portion, and a sensor loop; and
an electron paramagnetic resonance sensor material disposed within the sensor loop;
wherein the conductive wire loop is coated with a biocompatible plastic.

2. The implantable resonator and coupling device of claim 1 wherein the transmission line portion is twisted to form a twisted-pair transmission line portion.

3. The implantable resonator and coupling device of claim 1 wherein the transmission line portion comprises parallel wire.

4. The implantable resonator and coupling device of claim 1 wherein the conductive wire loop is further formed into a second sensor loop and a second transmission line portion.

5. The implantable resonator and coupling device of claim 4 wherein the conductive wire loop is further formed into a third sensor loop and a third transmission line portion.

6. The implantable resonator and coupling device of claim 4 wherein the conductive wire loop is further formed into a third transmission line portion, the third transmission line portion disposed between the coupling loop and an origin of the first and second transmission line portions.

7. The implantable resonator and coupling device of claim 4 wherein the coupling loop has a diameter of approximately between one-half centimeter and one and a half centimeters, and the sensor loop a diameter between one-half and one millimeter.

8. A system for measuring parameters in a living mammal or subject comprising:
a magnet for providing a nonuniform magnetic field in the mammal or subject;
a coupling device comprising a conductive wire loop of non-ferrous metal formed into a coupling loop, a twisted central portion, and at least a first and second sensor loop, and having an electron paramagnetic resonance sensor material disposed within each sensor loop; and
apparatus for measuring a radio frequency response coupled to a pickup coil, the pickup coil disposed near a skin surface of the mammal or subject and inductively coupled to the coupling loop, the apparatus for measuring a radio frequency response being capable of measuring a response of the electron paramagnetic resonance sensor material;
wherein the electron paramagnetic resonance sensor material is sensitive to a parameter of interest, and wherein the system is capable of measuring resonance for each of the first and second sensor loops individually by sweeping a system parameter selected from the group consisting of a strength of the magnetic field and a frequency of the radio frequency response.

9. The system of claim 8 wherein the electron paramagnetic resonance sensor material is selected from the group consisting of lithium Phtalocyanine, India ink, coals, charcoals, nitroxides, dithiocarbamates, nitrone compounds and nitroso compounds.

10. The system of claim 9 wherein the electron paramagnetic resonance sensor material is sensitive to pH.

11. The system of claim 9 wherein the electron paramagnetic resonance sensor material is sensitive to sulfhydryl concentration.

12. The system of claim 9 wherein the electron paramagnetic resonance sensor material is sensitive to membrane potential.

13. The system of claim 9 wherein the electron paramagnetic resonance sensor material comprises a paramagnetic material sensitive to oxygen concentrations.

14. The system of claim 9 wherein the electron paramagnetic resonance sensor material comprises a dithiocarbamate sensitive to nitric oxide concentrations.

15. The system of claim 8 wherein the electron paramagnetic resonance sensor material is coated with a gas permeable biocompatible plastic selected from the group consisting of fluorocarbon and dimethylsiloxane plastics.

16. The system of claim 8 wherein the system sweeps the strength of the magnetic field to measure resonance of the first and second sensor loops individually.

17. The system of claim 8 wherein the coupling device further comprises a third sensor loop, and having an electron paramagnetic resonance sensor material disposed within the third sensor loop; and wherein the system sweeps the strength of the magnetic field to measure resonance of the first, second, and third sensor loops individually.

18. A biopsy sampling device comprising:
a nonconductive outer needle having a conductive sensor loop attached thereto; and
a nonconductive central sampling needle for slideable engagement within the outer needle, the central sampling needle having a cavity for holding a sample of a biological material, the central sampling needle further comprising an electron paramagnetic resonance sensor material disposed adjacent to the cavity and coated with a gas-permeable biocompatible plastic;
wherein the sampling device has a first operative position wherein the central sampling needle is engaged within the outer needle with the electron paramagnetic resonance sensor material disposed near the conductive sensor loop and exposed to tissue, and wherein the cavity is exposed to tissue; and a second operative position wherein the cavity is not exposed to tissue.

19. The biopsy sampling device of claim 18 wherein the electron paramagnetic resonance sensor material comprises a paramagnetic material sensitive to oxygen concentrations.

20. The biopsy sampling device of claim 18 wherein the gas-permeable biocompatible plastic is selected from the group consisting of fluorocarbon and dimethylsiloxane plastics.

21. A coupling device for performing electron paramagnetic resonance of teeth comprising:
at least one nonconductive plastic chip for holding between teeth, the plastic chip having embedded therein a conductive wire sensor loop coupled to a first transmission line portion comprising two wires twisted together and extending from the plastic chip to a coupling loop.

22. The coupling device of claim 21 further comprising a second nonconductive plastic chip for holding between teeth, the second plastic chip having embedded therein a second conductive wire loop coupled to a second transmission line portion comprising two wires twisted together, the second transmission line portion electrically coupled to the coupling loop.

23. The coupling device of claim 22 wherein the second transmission line portion is electrically coupled to the coupling loop by connecting to an approximate midpoint of the first transmission line portion.

24. A coupling device for performing electron paramagnetic resonance of fingernails comprising:
a first and a second sensor loop, the first sensor loop electrically coupled to a first transmission line portion comprising two wires twisted together, the second sensor loop electrically coupled to a second transmission line portion, and the first and second transmission lines portions electrically coupled to a coupling loop; and
apparatus for retaining the first sensor loop near a first fingernail, and the second sensor loop near a second fingernail.
